(19) United States
(12) Patent Application Publication
(10) Pub. No.: US 2011/0206608 A1
(43) Pub. Date: Aug. 25, 2011

(54) Pharmaceutical Composition Of A Radioiodinated Benzamide Derivative And Methods Of Making Same

(76) Inventors: Jianqing Chen, Bordentown, NJ (US); James F. Kronauge, Cambridge, MA (US); John W. Babich, North Scituate, MA (US)

(21) Appl. No.: 13/062,410

(22) PCT Filed: Sep. 4, 2009
(86) PCT No.: PCT/US2009/056093

§ 371 Date: May 6, 2011

Related U.S. Application Data
(60) Provisional application No. 61/094,838, filed on Sep. 5, 2008.

(51) Int. Cl. A61K 51/04; C07C 237/30; A61P 35/00

(52) U.S. Cl. 424/1.85; 564/168

(57) ABSTRACT

Provided is a pharmaceutical composition comprising radioiodinated N-(2-(diethylamino)ethyl)-4-(4-fluorobenzamido)-5-iodo-2-methoxybenzamide of Formula I. The pharmaceutical composition provides a stable formulation for both storing and administering to patients having melanoma. Also provided is a novel method of iodinating the precursor compound.

### CROSS-REFERENCE TO RELATED APPLICATIONS

This application claims benefit of the provisional U.S. Application Ser. No. 61/094,838, filed 5 Sep. 2008, incorporated herein by reference in its entirety.

### BACKGROUND OF THE INVENTION

1. Field of the Invention

This invention is directed to a pharmaceutical composition containing radioiodinated benzamide derivative, N-(2-(diethylamino)ethyl)-4-(4-fluorobenzamido)-5-iodo-2-methoxybenzamide and a pharmaceutically acceptable excipient. The invention is also directed to methods of making the iodinated derivative and the pharmaceutical composition, as well as methods of treating a patient having a melanoma tumor using the pharmaceutical composition of the invention.

2. State of the Art

In the United States, there are currently 62,000 new cases of melanoma diagnosed annually. Melanoma has the fastest rising incident rate of any cancer and is particularly threatening because of the early and wide-spread metastases. Surgery to remove malignant melanoma is often not effective in most cases where the patient has a primary tumor of over three millimeters in diameter. Stage IV patients suffering from malignant melanoma have a 6-month life expectancy.

Between 75 and 90% of malignant melanomas contain melanin, a biopolymer containing indole units with carboxyl and phenolic hydroxy groups. Organic amines, metals, and polycyclic aromatic hydrocarbons are capable of binding to melanin. It has also been demonstrated that radiolabeled benzamide derivatives bind to melanin and exhibit high uptake and retention in melanoma cells, both in vitro and in vivo. See, for example, WO 2005/089815. In particular, it has been shown that the compound, N-(2-(diethylamino)ethyl)-4-(4-fluorobenzamido)-5-iodo-2-methoxybenzamide, has a particularly desirable ability to bind to melanin and is useful as an imaging agent and a therapeutic. See, the '815 publication.

Various difficulties arise when formulating radiolabeled drug products because of several reasons. First, there can be severe degradation of the product during the radioiodination process. Second, the drug product can often be difficult to formulate due to low solubility. Third, the radiolabeled drug product may not exhibit a high enough radiochemical yield suitable to treat the targeted disease. Further, the final radioactive product may not be stable at the concentration required for shipping, and the degradation products, such as free iodide, can cause serious life threatening cytotoxic effects to normal organs including the thyroid, endocrine organs and digestive system.

Various ¹²³I-benzamides have been exploited for diagnostic imaging of metastatic melanoma. Evidenced rapid tumor washout eliminated the possibility of using their ¹³¹I-labeled counterparts for therapeutic purpose. Some examples include:

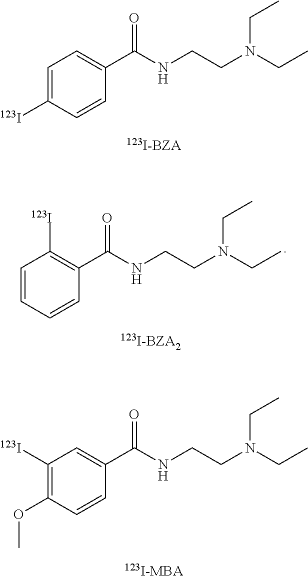

Example of rapid tumor wash-out (% ID/g) of ¹²³I-BZA in B16 melanoma bearing C57BL/J1 mouse model is disclosed in Moreau MF et al. Nucl Med Biol. 1995, 22: 737-47. The results are shown in Table A.

TABLE A
tumor wash-out (% ID/g) of ¹²³I-BZA

| 1 h | 24 h | 72 h |
|---|---|---|
| 6.8 | 0.8 | 0.3 |

Thus, there is a need for a substantially pure and stable drug product formulation containing therapeutics for treating melanoma.

### SUMMARY OF THE INVENTION

The invention is directed to pharmaceutical compositions useful for treating melanoma tumors. The pharmaceutical compositions of the invention may also be useful as diagnostics, i.e., for the purposes of imaging tumors.

In one embodiment, a pharmaceutical composition is provided comprising radioiodinated N-(2-(diethylamino)ethyl)-4-(4-fluorobenzamido)-5-iodo-2-methoxybenzamide of Formula I:

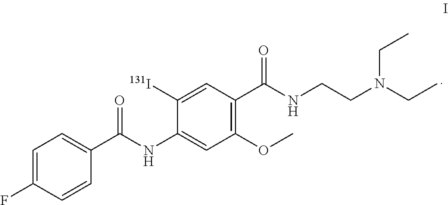

Also provided in the composition is at least one solubilizer and at least one preservative. The compositions of the invention have a pH of from about 4.0 to about 4.8. In another embodiment, the pH is about 4.4. The pH is selected such that the compositions maintain optimal stability and purity while being stored. In one embodiment, the compound of formula I is present in the composition in a concentration of about 1.25 mCi/milliter. In another embodiment, the composition comprises about 6% polyethylene glycol (w/v); about 2% ethanol (v/v); about 3% sodium gentisate (w/v); and about 6% ascorbic acid (w/v). In another embodiment, the ascorbic acid is about 3.7% sodium ascorbate and 2.7% ascorbic acid. In another embodiment, the composition further comprises N-(2-(diethylamino)ethyl)-4-(4-fluorobenzamido)-5-iodo-2-methoxybenzamide.

Also provided is a method for preparing a compound of formula III:

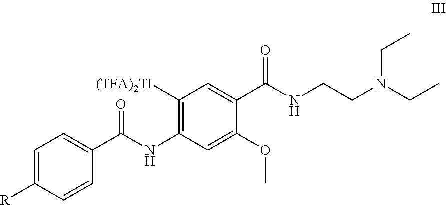

wherein R is fluoro, chloro or methoxy. The method comprises contacting under reaction conditions a compound of formula II:

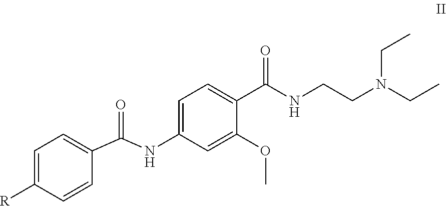

with a solution comprising at least about 1 equivalent of tris(2,2,2-trifluoroacetyl)thallium and a 1:1 volume of trifluoroacetic acid and acetic acid, to provide a compound of formula III.

In another aspect of the invention is provided a method of preparing a compound of formula IA:

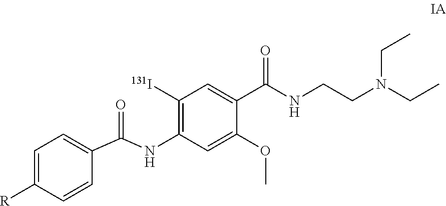

wherein R is fluoro, chloro or methoxy. The method comprises contacting under first reaction conditions a compound of formula II:

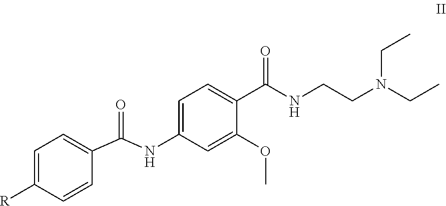

with a solution comprising at least about 1 equivalent of tris(2,2,2-trifluoroacetyl)thallium and about a 1:1 volume ratio of trifluoroacetic acid and acetic acid, to provide a compound of formula III:

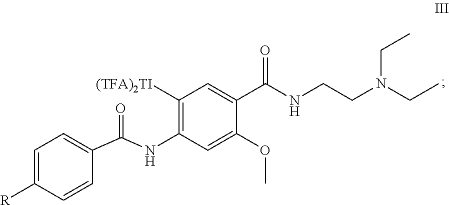

and

contacting under second reaction conditions a compound of formula III with a solution comprising at least about 0.5 to about 0.7 equivalents of sodium ¹³¹iodide, to provide a compound of formula I.

In one embodiment, the compound of formula II is present in a solution comprising acetic acid. In another embodiment (or the same embodiment just mentioned) the tris(2,2,2-trifluoroacetyl)thallium is present in a solution further comprising trifluoroacetic acid. In one embodiment, the sodium ¹³¹iodide is in a solution further comprising sodium hydroxide and sodium sulfate.

In one embodiment, the reaction conditions comprise a reaction time of about fifteen minutes at about 25 degrees Celsius. In another embodiment, the first reaction conditions comprise a reaction time of about ten minutes at about 25 degrees Celsius. In yet another embodiment, the second reaction conditions comprise a reaction time of about five minutes at about 25 degrees Celsius.

The methods of the invention also comprise isolating the compound of formula I (or IA). In one embodiment, the compound is isolated by high-performance liquid chromatography.

Also provided herein is a method for making the pharmaceutical composition of the invention. The steps for making the composition are found throughout the description and the attached appendix, which is hereby incorporated by reference. The compositions of the invention, when made by the methods of the invention have a radiochemical yield of about 70 to about 90% and a radiochemical purity of about 95% or greater when stored in a freezer for one week.

In one embodiment, compound of formula I has a specific activity of approximately 104 mCi/mg at least about one or two days after manufacture.

In another embodiment, the invention is directed to a method of treating a patient suffering from melanoma by administering a pharmaceutically effective amount of a pharmaceutical composition described herein.

### DETAILED DESCRIPTION OF THE INVENTION

Before the present compositions and methods are described, it is to be understood that the invention is not limited to the particular compounds, compositions, methodologies, protocols, and reagents described, as these may vary. It is also to be understood that the terminology used herein is intended to describe particular embodiments of the present invention, and is in no way intended to limit the scope of the present invention as set forth in the appended claims and examples.

### BRIEF DESCRIPTION OF THE DRAWINGS

Please note that Compound A is also referred to as “Cmpd A.” Compound B is also referred to as “Cmpd B” or “BA-52”. The degradation product of Compound

B is also referred to as “Cmpd BD” or “BA-52D.” Compound C is also referred to as “Cmpd C” or “MIP-1143.” Compound D is also referred to as “Cmpd D” or “MIP-1144.”

FIG. 1 illustrates whole body images of ¹³¹I-Cmpd B in melanoma patient at 120 h p.i. The first two panels show left and right views at one intensity setting while the third and fourth panels shows left and right views at another intensity setting;

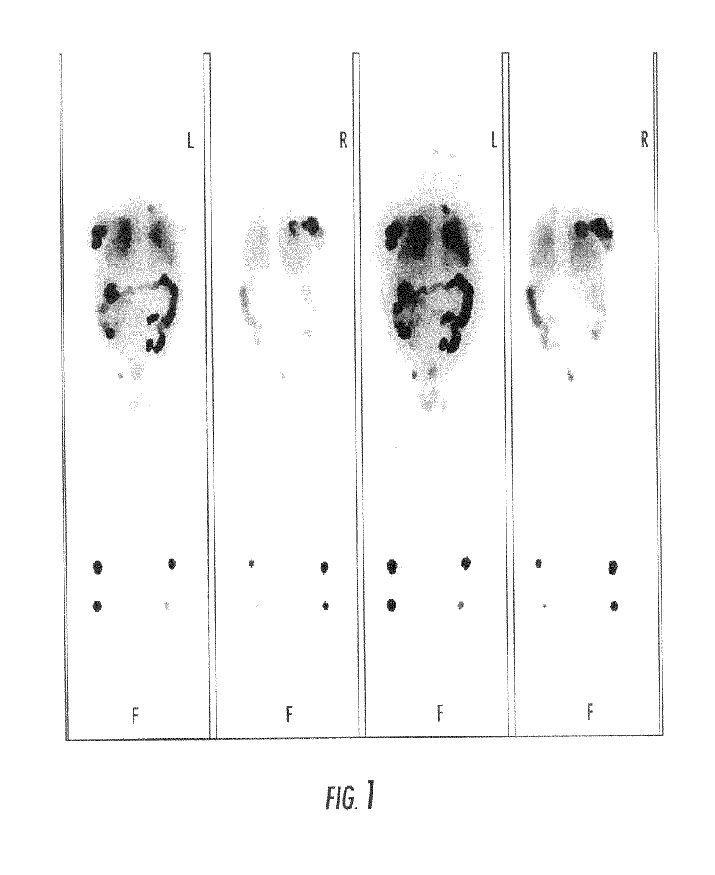

FIG. 2 illustrates the HPLC chromatogram and the reaction scheme for the by-product formed from ¹³¹I-Cmpd B. The structure of the by-product was confirmed by LC/MS;

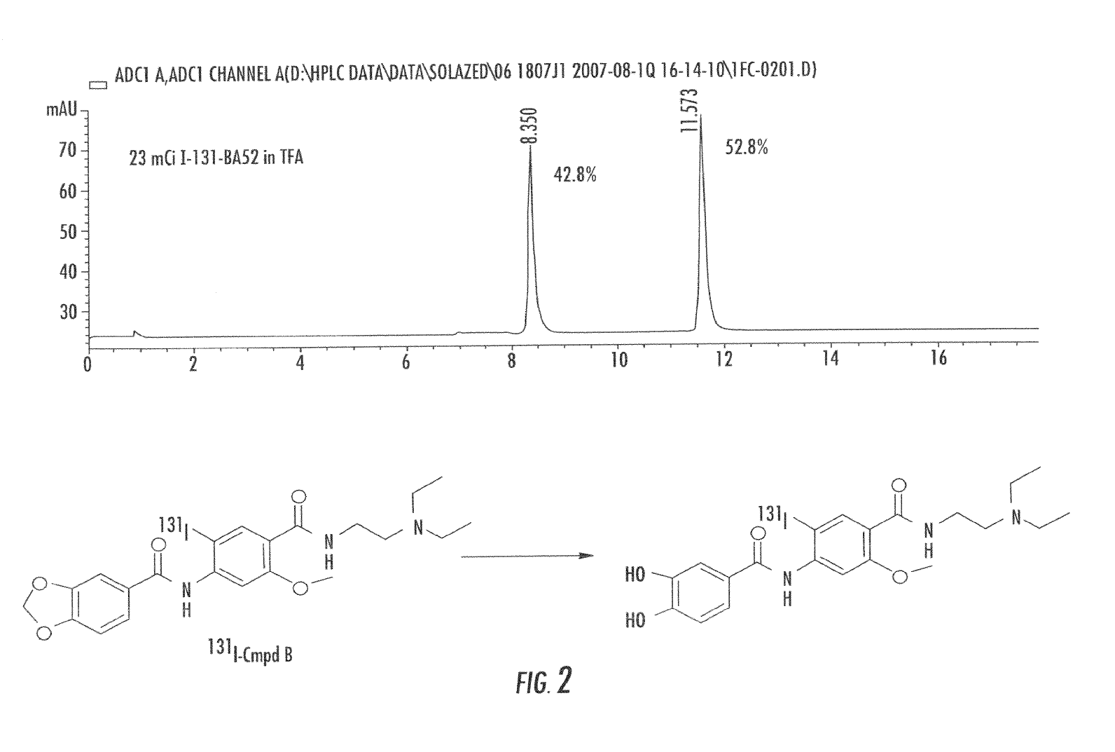

FIG. 3 illustrates the biodistribution of ¹³¹I-Cmpd B and ¹³¹I-Cmpd BD in B16F10 bearing mice. The graphs indicate low melanin targeting capacity for the by-product, ¹³¹I-Cmpd BD;

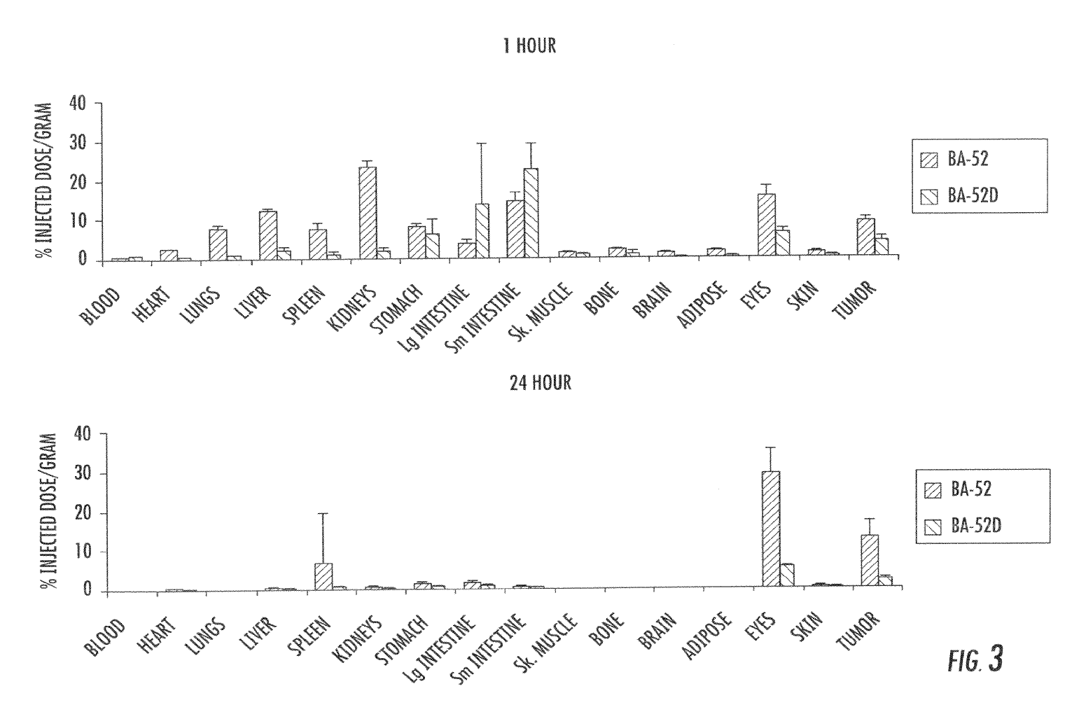

FIG. 4 illustrates the biodistribution of ¹³¹I-Cmpd A, ¹³¹I-Cmpd B, ¹³¹I-Cmpd C, and ¹³¹I-Cmpd D in B16F10 tumor bearing mice. The graphs indicate that desirable distribution properties are observed in all four tested compounds;

FIG. 5 illustrates the high stability of ¹³¹I-Cmpd A. The top two HPLC chromatograms indicate that the radioiodination of Cmpd A-p with 250 mCi of Na¹³¹I gives ¹³¹I-Cmpd A with an RCP>95% (specifically 96.5% and 96.2%). The bottom HPLC chromatogram indicates that the radioiodination of Cmpd B-p with 25 mCi of Na¹³¹I gives ¹³¹I-Cmpd B with an RCP˜65%;

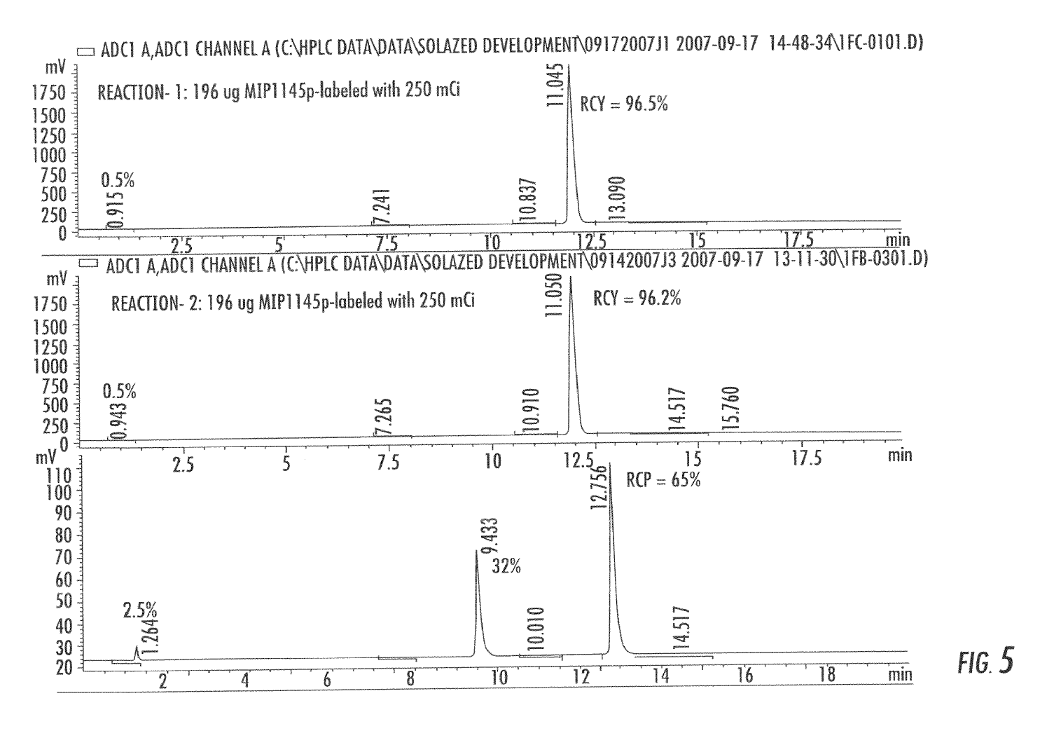

FIG. 6 illustrates the effect of ¹³¹I-Cmpd A (68 mCi/m²) on SK-MEL-3 tumor growth at various dose levels. The graph shows a plot of time versus tumor change with saline, dacarbazine×3, ¹³¹I-Cmpd A X1, ¹³¹I-Cmpd A X2, and ¹³¹-Cmpd A X3;

FIG. 7 illustrates the effect of ¹³¹I-Cmpd A (68 mCi/m²) on SK-MEL-3 tumor growth in mice at various dose levels. The graph shows a plot of elapsed time versus percent survival of the animals when treated with saline, dacarbazine×3, ¹³¹I-Cmpd A X1, ¹³¹I-Cmpd A X2, and ¹³¹I-Cmpd A X3. (Please note that animals were euthanized if tumor volume was>1500 mm³).

### DEFINITIONS

As used herein, certain terms may have the following defined meanings.

As used herein, the term “comprising” means that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the pharmaceutical composition. Embodiments defined by each of these transition terms are within the scope of the present technology.

In describing the details of the composition, the numerical ranges given herein are those amounts that provide the functional results in the composition. Thus, the ranges are generally introduced with the term “about” to indicate a certain flexibility in the range, i.e. ±10% or less at the lower and upper numerical ranges given.

The term “solubilizer” refers to a substance that is used to solubilize the compound of formula I. In one embodiment, the solubilizer is polyethylene glycol or “PEG”. As used herein, the term “polyethyleneglycol (PEG)” refers to a polyether of the formula —(OCH₂CH₂)ₙOH, wherein n can vary greatly depending on the composition. For example, the PEG can have a molecular weight of about 100 to about 1000 g/mol. In some embodiments of the invention, the PEG has a molecular weight of about 400 g/mol and is referred to herein as PEG400.

Another suitable solubilizer in the compositions of the invention is an alcohol of the formula R—OH, where R is a C₁ to C₄ hydrocarbyl radical that is straight-chained or branched. Examples include, but are not limited to, methanol, ethanol, and propanol. In one embodiment, the solubilizer is ethanol.

In some embodiments more than one solubilizer is present. For example, in one embodiment, both ethanol and PEG are present in the composition.

The term “preservative” refers to a substance that protects, prevents, or retards decay, discoloration, or other forms of spoilage under conditions of use or storage. Thus, a preservative may be one or more of an antioxidant, a chelator, an antibacterial, or the like. Suitable preservatives include sodium gentisate, methylparaben, butylparaben, propylparaben, benzyl alcohol, ascorbic acid, imidurea, thimerisal, propyl gallate, BHA, BHT, citric acid, disodium edetate, and the like. In one embodiment, sodium gentisate is employed in the composition.

In one embodiment of the invention, the preservative can also act as a buffer to adjust the pH of the composition to the desirable range of about 4.0 to about 4.8. In some embodiments, the pH is from about 4.1 to about 4.7. In one embodiment, the pH is about 4.4. A suitable preservative for this purpose is ascorbic acid. For example, about 3.7% sodium ascorbate and 2.7% ascorbic acid may be employed to achieve the desired pH of about 4.4.

In one embodiment, at least two preservatives are present. For example, in one embodiment, sodium gentisate, ascorbic acid and sodium ascorbate are present.

The term “Compound A” refers to N-(2-(diethylamino)ethyl)-4-(4-fluoro-benzoylamino)-5-iodo-2-methoxybenzamide.

The term “Compound A precursor” or “Compound A-p” refers to N-(2-(diethylamino)ethyl)-4-(4-fluorobenzoylamino)-2-methoxybenzamide.

The term “Compound B” refers to benzo[1,3]dioxole-5-carboxylic acid [4-(2-diethylamino-ethylcarbamoyl)-2-iodo-5-methoxyphenyl]amide.

The term “Compound B precursor” or “Compound B-p” refers to benzo[1,3]dioxole-5-carboxylic acid [4-(2-diethylamino-ethylcarbamoyl)-5-methoxyphenyl]amide.

The term “Compound C” refers to 4-(4-chlorobenzoylamino)-N-(2-(diethylamino)ethyl)-5-iodo-2-methoxybenzamide.

The term “Compound C precursor” or “Compound C-p” refers to 4-(4-chlorobenzoylamino)-N-(2-(diethylamino)ethyl)-2-methoxybenzamide.

The term “Compound D” refers to N-(2-diethylamino-ethyl)-5-iodo-2-methoxy-4-(4-methyoxybenzoylamino)benzamide.

The term “Compound D precursor” or “Compound D-p” refers to N-(2-(diethylamino)ethyl)-methoxy-4-(4-methyoxybenzoylamino)-benzamide.

The structures of compounds A-D are as shown below.

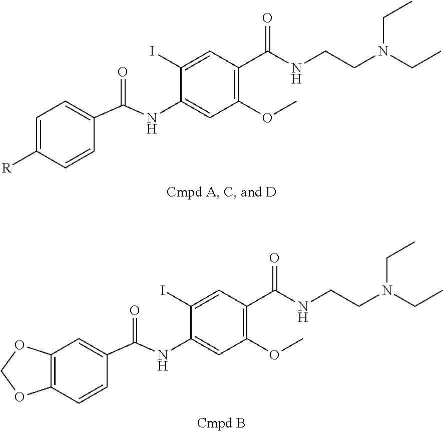

R

Cmpd A: F

Cmpd C: CL

Cmpd D: OMe

### Pharmaceutical Compositions

Compounds A, B, C, and D were all tested and found to have melanin targeting capacity both in vitro and in vivo. The results demonstrated that all the four tested compounds possessed a similar melanin targeting capacity and fast clearance from normal tissues/organs. Among them Compound A showed a significantly higher aqueous solubility and stability.

As illustrated in the examples, formulation testing of compound B evidenced degradation of the product during the radioiodination; the percentage of the degraded impurity was proportionally increased over the dose of the radioactivity applied. For instance, 42% of the applied radioactivity was turned into the degraded by-product in a formulation of Compound B with 23 mCi Na¹³¹I. The structure of the degraded impurity was identified by LC/MS, which was shown to be a by-product of oxidation of the dioxole moiety of Compound B into dihydroxy. Moreover, the results of biological testing showed that the melanin binding capacity of the degraded product was significantly lower than that of Compound B.

The Compound A formulation and the method of production is optimized as described herein. In one aspect, the radioiodination method was evaluated and optimized based on the modification of T1((TFA)₃/TFA iodination chemistry. By addition of the acetic acid to known iodination methods, the methods described herein can be used in dose-escalating studies. For instance, a radiochemical yield (RCY) of ˜90% is obtained in a 250 mCi dose level Compound A formulation without significantly changing the impurity profile. In another aspect, the component of the excipient was investigated and shown to enhance the solubility and stability of Compound A drug product. For example, the addition of 6% PEG400 (w/v) into the excipient significantly improves the solubility and recovery of Compound A. In yet another aspect, an HPLC purification method was evaluated for Compound A drug purification from remaining precursor and any possible chemical and radioactive impurities existing in the Compound A crude reaction solution.

In summary, as demonstrated throughout, (1) the excipient formulation of Compound A exhibits a higher chemical stability than other derivatives; (2) Compound A is preferred based on superior solubility, stability, melanin targeting capacity, as well as desirable distribution properties in tumor bearing animal models; (3) a robust production and purification process for Compound A is provided; (4) a formulation enhancement to increase the stability and shelf life for Compound A is provided; (5) storage condition and impurity profile for Compound A drug product is also provided. In conclusion, Compound A drug product (1.25 mCi/mL at TOC) can be produced in an overall RCY of 70-95% with an RCP of >95% (free I-131<5%) over one week storage in the frozen state (TOE).

### Method for Making Compound of Formula I and Pharmaceutical Compositions Comprising Formula I

It is further contemplated that the methods provided herein can be used to radioiodinate Compounds A, C, and D. Previous methods taught in the art employ trifluoroacetic acid (TFA) and tris(2,2,2-trifluoroacetyl)thallium. The degradation of both precursor and product in trifluoroacetic acid (TFA) solution may occur over time due to the severe corrosiveness of TFA. Therefore, it is contemplated that dissolving the precursor in acetic acid prior to the iodination improves the stability and/or yield. Further, due to the low melting and boiling points of TFA, it can readily evaporate, especially when using a small volume. This evaporation potentially impacts the reproducibility of the labeling. However, it is contemplated that adding acetic acid overcomes this problem. Use of acetic acid is considered to be even more valuable in large dose iodination processes, such as in the case of iodinating drugs for therapeutic purposes. Typically in therapeutics, a radiolabeled drug is produced in approximately 3 Curie per batch, and an automation system is often employed for this purpose. The TFA evaporation is problematic in automation systems which often employ solution transfer techniques. It is contemplated that adding acetic acid increases the reaction volume for T1-complex formation without increasing the level of degradation.

Therefore, in one embodiment of this invention is provided a method of making a compound of formula I:

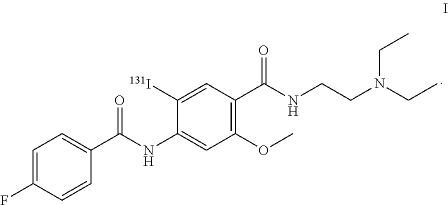

The compound can be synthesized according to the Scheme A:

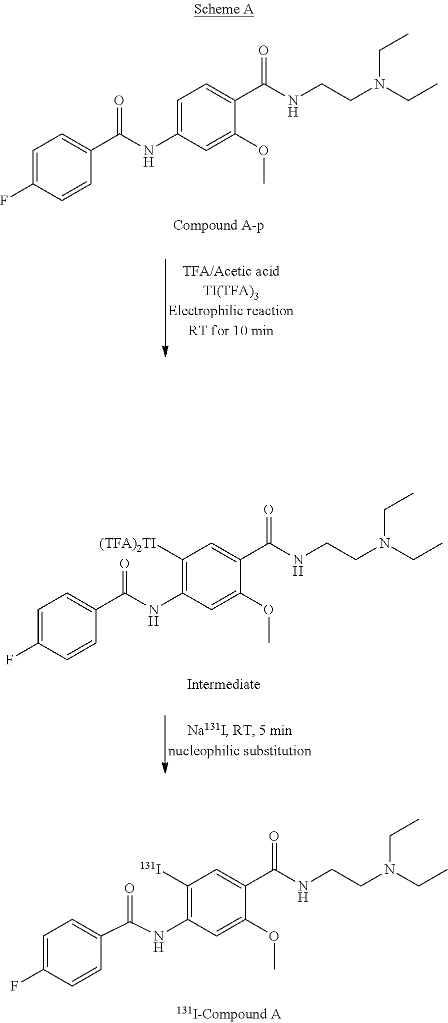

Prior to providing the specifics of the reaction, it should be noted that all abbreviations may be found in the example section.

Eighty micrograms of Compound A precursor (Compound A-p, dissolved in acetic acid at 5 mg/mL) were mixed with T1(TFA)₃ (dissolved in TFA at 10 mg/mL) in a molar ratio of about 1 to 1.2. The solution was brought to a final volume of 300 μL in 50% acetic acid/50% TFA (v/v). After a 10-minute incubation of the mixture at RT, the solution was transferred into the 2-mL Na¹³¹I source vial containing 50-100 mCi I-131 in ˜50 μL of 0.1 N NaOH/0.02 M Na₂SO₄. The reaction solution was mixed and allowed to incubate at RT for an additional 5 minutes.

The crude reaction was diluted in 1.5 mL excipient (6% PEG400 (w/v), 2% ethanol (v/v), 6% ascorbic acid (w/v), and 3% sodium gentisate (w/v), pH 4.4), and the product was purified by RP-HPLC with a C18 column. The compound was eluted with a gradient of 25-60% water (buffer B) over 10 minutes at a flow rate of 2 mL/min using 2.5% ascorbic acid (w/v)/0.5% acetic acid (v/v) in water (buffer A) and 2.5% ascorbic acid (w/v)/85% ethanol (v/v) in buffer B as the solvents. The product peak was collected into a 30-mL vial and the volatile organics in the collected solution were removed by heating the vial at 70 ° C. under vacuum/Nitrogen gas stream for 30 minutes.

Additional Compound A (to an amount of 44 μg per patient dose, 5 mCi at TOC) was added into the bulk formulation container. The Compound A formulation was diluted in excipient with a final radioactive concentration of 1.25 mCi/mL in a specific activity of approximately 104 mCi/mg at TOC. The final product solution was sterilized by passing through a sterile 0.2 μm Millex GV syringe filter, and then aseptically dispensed into sterile and pyrogen free 2 mL vials. The target RCP of the product is ≧90% with a free I-131≦5% at TOE.

The synthesis just provided can be readily adapted to iodinate Compounds C and D.

¹³¹I-Compound B is also synthesized in an analogous manner as shown in

Scheme B below.

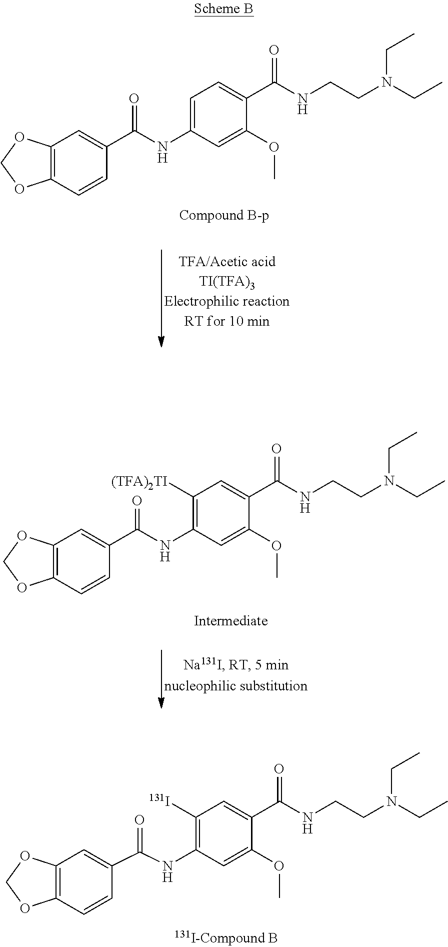

### Methods for Treating Melanoma

As mentioned above, radioiodinated benzamide derivatives specifically binding to melanin showed their potential of being used as molecular targeting imaging agents for melanoma diagnosis; while the fast washing out of the tumor impacted them being used in therapeutic purpose. However, significant high tumor uptake and prolonged retention is evidenced for ¹³¹I-Compound A, N-(2-diethylamino-ethyl)-4-(4-fluorine-benzamido)-5-iodo-2-methoxy-benzamide. ¹³¹I-Compound A has demonstrated superiority in stability, solubility, melanin target capacity, as well as desirable distribution properties in tumor bearing animal models compared to other tested compounds. Moreover, complete response (CR) has been observed for ¹³¹I-Compound A treatment (68 mCi/m²) in a human melanoma mouse xenograft model.

Accordingly, one embodiment of the invention is directed to a method of treating a patient suffering from melanoma comprising administering to said patient a pharmaceutically effective amount of a composition of the invention.

The term “treatment” or “treating” means any treatment of a disease or disorder in a subject, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

A composition of this invention may be administered to a mammal by a suitable route, such as orally, intravenously, parenterally, transdermally, topically, rectally, or intranasally. In one embodiment, the composition is administered intravenously.

Mammals include, for example, humans and other primates, pet or companion animals, such as dogs and cats, laboratory animals, such as rats, mice and rabbits, and farm animals, such as horses, pigs, sheep, and cattle. In one embodiment, the mammal is human.

Tumors or neoplasms include growths of tissue cells in which the multiplication of the cells is uncontrolled and progressive. Some such growths are benign, but others are termed “malignant” and can lead to death of the organism. Malignant neoplasms or “cancers” are distinguished from benign growths in that, in addition to exhibiting aggressive cellular proliferation, they can invade surrounding tissues and metastasize. Moreover, malignant neoplasms are characterized in that they show a greater loss of differentiation (greater “dedifferentiation”) and organization relative to one another and to surrounding tissues. This property is called “anaplasia.”

The compositions administered to a patient are typically in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. It will be understood that use of certain of the foregoing excipients, carriers, or stabilizers will result in the formation of pharmaceutical salts.

The therapeutic dosage of the compounds and/or compositions of the present invention will vary according to, for example, the particular use for which the treatment is made, the manner of administration of the compound, the health and condition of the patient, and the judgment of the prescribing physician. For example, for oral administration, the dose will typically be in the range of about 5 μg to about 50 mg per kilogram body weight per day, preferably about 1 mg to about 10 mg per kilogram body weight per day. In the alternative, for intravenous administration, the dose will typically be in the range of about 5 μg to about 50 mg per kilogram body weight, preferably about 500 μg to about 5000 μg per kilogram body weight. Alternative routes of administration contemplated include, but are not limited to, intranasal, transdermal, inhaled, subcutaneous and intramuscular. Effective doses can be extrapolated from dose-response curves derived from in vitro or animal model test systems.

The amount administered to the patient will vary depending upon what is being administered, the purpose of the administration, therapy, the state of the patient, the manner of administration, and the like. In therapeutic applications, compositions are administered to a patient already suffering from a disease in an amount sufficient to cure or at least partially arrest the progression or symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the disease, disorder or condition, the age, weight and general condition of the patient, and the like.

In general, the compositions of the subject invention will be administered in a therapeutically effective amount by any of the accepted modes of administration for agents that serve similar utilities. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound and/or composition used in the method of the invention, the therapeutically effective dose can be estimated initially from cell culture assays. A dose may be formulated in animal models to achieve a circulating plasma concentration range which includes the IC₅₀ (the concentration of the test compound which achieves a half-maximal inhibition of activity) as determined in cell culture. Such information can be used to more accurately determine useful doses in humans. Levels in plasma may be measured, for example, by high performance liquid chromatography.

Tumor uptake and retention of ¹²³I-Compound B in B16 melanoma bearing mice (% ID/g) was as shown in Table B. Those studies were done using the method disclosed in Eisenhut et al., J. Med. Chem. 2000, 43(21), 3913-22.

TABLE B
uptake and retention of ¹²³I-Compound B

| 1 h | 5 h | 48 h |
|---|---|---|
| 14.8 | 22.7 | 16.6 |

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

Unless otherwise stated all temperatures are in degrees Celsius (° C.). Also, in these examples and elsewhere, abbreviations have the following meanings:

| | |
|---|---|
| Compound A | N-(2-(diethylamino)ethyl)-4-(4-fluorobenzoylamino)-5-iodo-2- |
| | methoxybenzamide |
| Compound A | N-(2-(diethylamino)ethyl)-4-(4-fluorobenzoylamino)-2- |
| precursor | methoxybenzamide |
| (Compound A-p) | |
| Compound B | Benzo[1,3]dioxole-5-carboxylic acid [4-(2-diethylamino- |
| | ethylcarbamoyl)-2-iodo-5-methoxyphenyl]amide |
| Compound B | Benzo[1,3]dioxole-5-carboxylic acid [4-(2-diethylamino- |
| precursor | ethylcarbamoyl)-5-methoxyphenyl] amide |
| (Compound B-p) | |
| Compound C | 4-(4-chlorobenzoylamino)-N-(2-(diethylamino)ethyl)-5-iodo-2- |
| | methoxybenzamide |
| Compound C | 4-(4-chlorobenzoylamino)-N-(2-(diethylamino)ethyl)-2- |
| precursor | methoxybenzamide |
| (Compound C-p) | |
| Compound D | N-(2-(diethylamino)ethyl)-5-iodo-2-methoxy-4-(4- |
| | methyoxybenzoylamino)-benzamide |
| Compound D | N-(2-(diethylamino)ethyl)-methoxy-4-(4- |
| precursor | methyoxybenzoylamino)-benzamide |
| (Compound D-p) | |
| HPLC | High Performance Liquid Chromatography |
| C18 | ZORBAX Eclipse Plus C18 Column (4.6 × 100 mm, 5-μm), Agilent |
| Sep-Pak C18 | Sep-Pak ® Plus C18 Cartridge, Waters |
| Ci | Curie |
| mCi | Millicurie |
| I-131 | Iodine-131 radioisotope |
| RCP | Radiochemical Purity |
| RCY | Radiochemical Yield |
| N/A | Not Applicable |
| PEG400 | Polyethylene Glycol 400 |
| TFA | Trifluoroacetic acid |
| Tl(TFA)₃ | Thallium trifluoroacetate |
| RT | Room Temperature |
| SOP | Standard Operating Procedure |
| TOM | Time of manufacture |
| TOC | Time of calibration (2 days post TOM) |
| TOE | Time of expiry (7 days post TOM) |
| μg | Micrograms |
| μL | Microliters |
| min | Minutes |
| RT | Room temperature |
| mg | Milligram |
| mL | Milliliter |
| mm | Millimeter |
| μm | Micrometer |
| ACN | Acetonitrile |
| mw | Molecular weight |
| N | Normal |
| mM | Millimolar |
| M | Molar |
| w/v | weight to total volume |
| v/v | volume to total volume |
| RP-HPLC | reverse phrase high performance liquid chromatography |
| hrs | hours |
| LC/MS | liquid chromatography mass spectroscopy |
| tem. | temperature |
| Et₂O | diethyl ether |
| SWFI | sterile water for injection |

### Example 1

### Instability of Compound B During the Iodination Procedure

Purpose: Test the reproducibility of Compound B iodination method described in the art; the incubation time for intermediate formation and iodination was further evaluated.

### Example 1-A

### Compound B Iodination With Small Scale of Na¹³¹I (˜1 mCi)

TABLE 1-1
Experimental conditions
Step-1: Intermediate formation

| | |
|---|---|
| Compound B-p | 80 μg in TFA |
| Tl(TFA)₃ | 40 μg in TFA |
| Total reaction volume in TFA | 150 μL |
| Incubation time for | 5 min at RT |
| intermediate | |

Step-2: Radioiodination

| | |
|---|---|
| mCi of Na¹³¹I | 1.8 mCi |
| Volume of Na¹³¹I source | 20 μL |
| solution | |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 92-94% (n = 2) |
| Degraded impurity | ~6% |
| Free I-¹³¹ | <0.5% |

Results: Compound B labeling in T1(TFA)₃/TFA iodination chemistry was successfully conducted at a radioactivity dose level of ˜2 mCi. An RCP of >90% with a free ¹³¹I<1% was obtained for the labeling.

### Example 1-B

### Effect of Incubation Time of the Intermediate Formation (2, 5, 10, 20 and 30 min) on the Yield of Compound B Labeling

TABLE 1-2
Experimental conditions
Step-1: Intermediate formation

| | |
|---|---|
| Compound B-p | 80 μg in TFA |
| Tl(TFA)₃ | 40 μg in TFA |
| Total reaction volume in TFA | 150 μL |
| Incubation time for | 2, 5, 10, 20 & 30 min at RT |
| intermediate formation | |

Step-2: Radioiodination

| | |
|---|---|
| mCi of Na¹³¹I | ~0.5 mCi (3.75 μg Na¹²⁷I) |
| (mixture of ¹³¹I/¹²⁷I) | |
| Volume of the radioactivity | 15 μL |
| Reaction Vial | 2-mL Hollister |
| Reaction time | 5 min at RT |
| Volume of protecting | 0.1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 81.4~97.7% (see Table 1-3) |
| Degraded impurity | 1.5~17.9% (see Table 1-3) |
| Free I-131 | <0.5% |

Results: The formation of the intermediate could be completed within 2 minutes of incubation at RT; prolonged incubation would increase the degraded product due to the character of strong oxidant of T1(TFA)₃. The data is listed in Table 1-3.

TABLE 1-3
The effect of incubation time of thallation
at RT on the yield of Compound B

| | 2 min | 5 min | 10 min | 20 min | 30 min |
|---|---|---|---|---|---|
| RCP (%) | 97.7 | 95.5 | 91.5 | 88.1 | 81.4 |
| Degraded | 1.5 | 4.0 | 8.0 | 10.8 | 17.9 |
| Impurities (%) | | | | | |

### Example 1-C

### Effect of Incubation Time of the Iodination (varied for 1, 2, 5 and 10 min) on the Yield of Compound B

TABLE 1-4
Experimental conditions
Step-1: Intermediate formation

| | |
|---|---|
| Compound B-p | 80 μg in TFA |
| Tl(TFA)₃ | 40 μg in TFA |
| Total reaction volume in TFA | 150 μL |
| Incubation time for | 5 min at RT |
| intermediate | |

Step-2: Radioiodination

| | |
|---|---|
| mCi of Na¹³¹I | ~0.5 mCi (3.75 μg Na¹²⁷I) |
| (mixture of ¹³¹I/¹²⁷I) | |
| Volume of the radioactivity | 20 μL |
| Reaction Vial | 2-mL Hollister |
| Reaction time | 1, 2, 5, & 10 min at RT |
| Volume of protecting | 0.1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 83.5~85.1% (see Table 1-5) |
| Degraded impurity | 1.5~17.9% (see Table 1-5) |
| Free I-131 | <0.5% |

Results: The iodination of Compound B could be completed within 1 min incubation at RT; the prolonged incubation time would not significantly impact the RCY. The data is listed in Table 1-5.

TABLE 1-5
The effect of iodination time at RT
on the yield of Compound B labeling

| | 1 min | 2 min | 5 min | 10 min |
|---|---|---|---|---|
| RCP (%) | 83.7 | 83.5 | 83.6 | 85.1 |
| Impurities (%) | 10.4 | 11.4 | 10.4 | 9.7 |

### Example 1-D

### Degradation Increased Over the Radioactivity Dose Applied in Compound B Labelling

TABLE 1-6
Experimental conditions
Step-1: Intermediate formation

| | |
|---|---|
| Compound B-p | 80 μg in TFA |
| Tl(TFA)₃ | 40 μg in TFA |
| Total reaction volume in TFA | 150 μL |
| Incubation time for | 5 min at RT |
| intermediate | |

Step-2: Radioiodination

| | |
|---|---|
| mCi of Na¹³¹I | 2.2 or 23 mCi |
| Volume of the radioactivity | 40 μL |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 89.5~54.8% (see Table 1-7) |
| Degraded impurity | 5.8~42.8% (see Table 1-7) |
| Free I-131 | <0.5% |

Results: The data that the percentage of the degraded impurity was increased over the radioactivity dose of ¹³¹I applied in Compound B labeling. The comparison in degraded product for Compound B labeled with 2.2 or 23 mCi Na¹³¹I is listed in Table 1-7.

TABLE 1-7
The degraded by-product increased over the radioactivity
dose applied in Compound B labeling

| | 2.2 mCi | 23 mCi |
|---|---|---|
| RCP (%) | 89.5 | 54.8 |
| Degraded | 5.8 | 42.8 |
| impurity (%) | | |

The degraded impurity was identified as degradation product of the dioxolane moiety into dihydroxyl form for both precursor (Compound B-p) and final product (Compound B) as discussed in Example 2.

Based on the experiments above, Compound B could be formed via T1(TFA)₃/TFA chemistry. The intermediate of T1(TFA)₂-Compound B complex could be completed within a 5 minutes incubation at RT, and increasing of the incubation time would increase the degradation of the drug product owing to the nature of oxidation of T1(TFA)₃. The nucleophilic substitution of ¹³¹I from T1(TFA)₂ could be completed within 5 minutes incubation at RT in Compound B labeling. A poor chemical stability of Compound B was evidenced, and the level of degradation was significantly increased over the radioactivity dose applied in Compound B labeling.

### Example 2

### Identification of the Degraded By-Product of Compound B by LC/MS

The purpose of these examples is to identify the observed main degraded by-product of Compound B by LC/MS.

TABLE 2-1
Experimental conditions
Step-1: Intermediate formation

| | |
|---|---|
| Compound B-p | 80 μg in TFA |
| Tl(TFA)₃ | 40 μg in TFA |
| Total reaction volume in TFA | 150 μL |
| Incubation time for | 5 min at RT |
| intermediate | |

Step-2: Radioiodination

| | |
|---|---|
| mCi of Na¹³¹I | ~0.5 mCi (3.75 μg Na¹²⁷I) |
| (mixture of ¹³¹I/¹²⁷I) | |
| Volume of the radioactivity | 20 μL |
| Reaction Vial | 2-mL Hollister |
| Reaction time | 5 min at RT |
| Volume of protecting | 0.1 mL (6 mg/mL sodium Ascorbate) |
| solution added | |
| LC/MS analysis | Eclipse plus C18 (4.6 × 150 mm; |
| | 3.5 μm) |
| | Gradient: 10-50% B@15 min |
| | Solvent A: 0.1% Formic acid/H₂O; |
| | Solvent B: 0.1% Formic acid/ACN |
| | Flow rate: 1.5 mL/min |
| RCP | 91.5% |
| Degraded impurity | 8.0% |
| Free I-131 | <0.5% |

Results: The degraded impurity has been identified by LC/MS, and the data is listed in Table 2-2. The results demonstrated that the degradation of the dioxolane moiety into dihydroxyl form occurred for both precursor (Compound B-p) and final product (Compound B).

TABLE 2-2
Degraded impurities for both Compound
B-p and Compound B by LC/MS

| | Precursor | | Product | |
|---|---|---|---|---|
| | Compound | Degraded | | Degraded |
| | B-p | form | Compound B | form |
| Retention time | 4.5 min | 6.2 min | 5.7 min | 7.5 min |
| on LC | | | | |
| Expected mw | 413.2 | 401.2 | 539.1 | 527.1 |
| Measured (M + H⁺) | 414.2 | 402.2 | 540.2 | 527.9 |

### Example 3

### Efforts to Enhance the Stability of Compound B

The purpose of this example was to investigate the possibility of using alternative iodination methods in Compound B radioiodination to decrease the degradation and consequently enhance the RCY.

### Example 3-A

### ¹²⁷I-Compound B iodinated in KIO₃ Iodination Method

TABLE 3-1
Experimental conditions

| | |
|---|---|
| Compound B-p | 80 μg in 0.1N HCl |
| KIO₃ | 7.5 μg, 50 mM in 0.1N HCl |
| Total reaction volume | 100 μL |
| in 0.1N HCl | |
| Na¹²⁷I | 8 μg |
| Reaction Vial | 2-mL Hollister |
| Reaction time | 20 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | No labeling |

### Example 3-B

### ¹²⁷I-Compound B iodinated in T1(TFA)₃/BF₃.Et₂.O Iodination Method

TABLE 3-2
Experimental conditions

| Compound B-p | 80 μg in CH₂Cl₂ |
|---|---|
| Tl(TFA)₃ | 80 μg in CH₂Cl₂ |
| Total reaction volume | 190 μL |
| in CH₂Cl₂ | |
| Na¹²⁷I | 10 μg |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 10% |

Results: The RCY was low with an overall RCY of approximately 10%. Moreover, a new form of impurity was observed in this iodination method.

### Example 3-C

### Compound B Labeled by Using Tin-Precursor of Compound B

TABLE 3-3
Experimental conditions

| Compound B-Tin precursor | ~50 μg in Methanol |
|---|---|
| Na¹³¹I | 20 μL, 13.2 mCi |
| 4.5% H₂O₂ (0.85 mL acetic | 50 μL |
| acid/0.15 mL H₂O₂) | |
| Total reaction volume | 120 μL |
| Reaction time | 10 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 86.1% |
| Impurity | 13.9% |
| Free I-131 | <0.5% |

Results: The reaction could be completed within 10 min incubation at RT.

However, there was an impurity peak (13.9%) with a retention time of 6.9 minutes under this described analytic HPLC condition. None of the tested alternative iodination methods showed significant benefit of being used in Compound B radioiodination compared to T1(TFA)₃/TFA method.

### Example 4

### Efforts of Using Protecting Agent to Decrease the Degradation of Compound B

Purpose: Adding protecting agents, dioxolane and trioxane, in T1(TFA)₃/TFA iodination solution to enhance the yield of Compound B by decreasing the formation of the degraded by-product.

### Example 4-A

### Protecting Effect of Dioxolane on Compound B Formation

TABLE 4-1
Experimental conditions
Step-1: Intermediate formation

| | |
|---|---|
| Compound B-p | 80 μg in TFA |
| Tl(TFA)₃ | 80 μg in TFA |
| Total volume of TFA | 150 μL |
| 1,3-Dioxolane | 20 μL |
| Incubation time for | 5 min at RT |
| intermediate | |

Step-2: Radioiodination

| | |
|---|---|
| mCi of Na¹³¹I | 2.2 or 23 mCi |
| Volume of the radioactivity | 5 μL |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 84.8-92.4% |
| Identified impurity | 1.1-11.6% |
| Free I-131 | <0.5% |

Results: Compared to the control (without adding dioxolane), the addition of dioxolane into the reaction solution could significantly reduced the degradation of Compound B. The data is listed in Table 4-2. This result indirectly confirmed that the degradation of Compound B was due to the oxidation of the dioxolane moiety of the drug product into diohydroxyl because of the nature of oxidant of T1(TFA)₃.

TABLE 4-2
Adding Dioxolane to significantly improve the yield
of Compound B labeled with 2.2 or 23 mCi of Na¹³¹I

| | |
|---|---|
| 2.2 mCi | 23 mCi |

| | Control | Dioxolane | Control | Dioxolane |
|---|---|---|---|---|
| RCP (%) | 89.5 | 92.4 | 54.8 | 84.8 |
| Identified | 5.8 | 1.1 | 42.8 | 11.6 |
| impurity (%) | | | | |

### Example 4-B

### Protecting Effect of Trioxane on Compound B Formation

TABLE 4-3
Experimental conditions
Step-1: Intermediate formation

| | |
|---|---|
| Compound B-p | 80 μg in TFA |
| Tl(TFA)₃ | 80 μg in TFA |
| Total volume of TFA | 150 μL |
| 1,3,5-Trioxane | 20 μL |
| Incubation time for | 5 min at RT |
| intermediate | |

Step-2: Radioiodination

| | |
|---|---|
| mCi of Na¹³¹I | 2.2 |
| Volume of the radioactivity | 5 μL |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 86.6% |
| Identified impurity | 2.7% |
| Free I-131 | <0.5% |

Results: The addition of trioxane into T1(TFA)₃/TFA iodination reaction solution could also increase the yield of Compound B and decrease degradation. The addition of either dioxolane or trioxane into T1(TFA)₃/TFA iodination reaction solution could significantly reduce the degradation of Compound B, and as a consequence enhance the RCY. This result indirectly confirmed that the degradation of Compound B was due to the oxidation of the dioxolane moiety of the drug product into diohydroxyl because of the nature of oxidant of T1(TFA)₃. The protecting efficiency might not be high enough to be used in therapeutic dose Compound B radioiodination, although addition of 20 μL dioxolane could decrease the degradation from 42.8% to 11.6% in 23 mCi Compound B labeling.

### Example 5

### Comparison the Stability of Compound B Iodinated by Use of Different Batches of Precursor (Compound B-p)

Purpose: The confirmation of the generation of degraded impurity during the iodination of Compound B was conducted by using two different batches of Compound B-p produced by different vendors (Cambridge major and University of Heidelberg with catalogue numbers of 140-0124 and 140-0114, respectively).

TABLE 5-1
Experimental conditions:
Step-1: Intermediate formation

| | |
|---|---|
| Compound B-p | 80 μg in TFA |
| Tl(TFA)₃ | 80 μg in TFA |
| Total volume of TFA | 150 μL |
| Incubation time for | 5 min at RT |
| intermediate | |

Step-2: Radioiodination

| | |
|---|---|
| mCi of Na¹³¹I | 0.9 mCi |
| Volume of the radioactivity | 5 μL |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| RCP | 86.2%; 87.6% |
| Identified impurity | 4.7%; 4.1% |
| Free I-131 | <0.5% |

Results: There was no significant difference in RCY and percentage of identified degraded impurity for Compound B labeled in two different batches of Compound B precursor. The data of the comparison is listed in Table 5-2.

TABLE 5-2
The degraded impurity observed in Compound B labeled (with 0.9
mCi of Na¹³¹I) by using two different batches of Compound B-p

| | Compound B-p | Compound B-p |
|---|---|---|
| RCP (%) | 87.6 | 86.2 |
| Identified | 4.1 | 4.7 |
| impurity (%) | | |

### Example 6

### Comparison of the Stability of Compound C, Compound D & Compound A in ¹³¹I-labeling

Purpose: ¹³¹I-labeling yield and stability were compared side-by-side for (Compound C, Compound D and Compound A) together with Compound B (with or without addition of Dioxolane).

### Example 6-A

### ¹³¹I-labeling for Compound C, Compound D, Compound A and Compound B.

TABLE 6-1
Experimental conditions
Step-1: Intermediate formation

| | |
|---|---|
| Compound B-p | 80 μg in TFA |
| Tl(TFA)₃ | 80 μg in TFA |
| Total volume of TFA | 150 μL |
| Incubation time for | 5 min at RT |
| intermediate | |

Step-2: Radioiodination

| | |
|---|---|
| mCi of Na¹³¹I | 2.5 |
| Volume of the radioactivity | 10 μL |
| Reaction Vial | 2-mL Hollister |
| Reaction time | 5 min at RT |
| Volume of protecting | 1 mL (6 mg/mL sodium Ascorbate) |
| solution added | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tern: 30° C. |
| | Flow rate: 1.5 mL/min |
| RCP | 90.3~100% |
| Identified impurity | 0~5.5% |
| Free I-131 | <0.5% |

Results: The T1(TFA)₃/TFA iodination method could be used for ¹³¹I-Compound C, ¹³¹I-Compound D and Compound A. Different from Compound B, there was almost no degraded impurities occurred during the labeling (¹³¹I-Compound C, ¹³¹I-Compound D or Compound A) at a radioactivity dose level of 2.5 mCi. The data of the comparison is listed in Table 6-2.

TABLE 6-2
There was no degradation observed in I-labeling for Compound C,
Compound D and Compound A in Tl(TFA)3/TFA iodination chemistry

| | ¹³¹I- | ¹³¹I- | | | |
|---|---|---|---|---|---|
| | Compound C | Compound D | Compound A | Compound B | Compound B* |
| RCP (%) | 97.1 | ~100 | ~100 | 90.3 | 94.0 |
| Identified | 0 | 0 | 0 | 5.5 | 2.3 |
| impurity (%) | | | | | |

*Protected with 10 μL of Dioxolane

### Example 6-B

### Stability Comparison Between Compound A and Compound B Post Labelling and Protected by Adding Radiolysis Protecting Buffer

TABLE 6-3
Experimental conditions
Step-1: Intermediate formation

| | |
|---|---|
| Compound B-p | 80 μg in TFA |
| Tl(TFA)₃ | 80 μg in TFA |
| Total volume of TFA | 300 μL |
| Incubation time for | 5 min at RT |
| intermediate | |

Step-2: Radioiodination

| | |
|---|---|
| mCi of Na¹³¹I | 30 |
| Volume of the radioactivity | 20 μL |
| Reaction Vial | 2-mL Hollister |
| Reaction time | 5 min at RT |
| Volume of protecting | 1 mL |
| solution added* | |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |

*Protecting solution: 10% ascorbic acid, 5.6% sodium gentisate, 6% PEG400, pH = 3.4

PEG400, pH=3.4

Results: There was no significant difference in stability between Compound B and Compound A during the 3 days storage at RT post labeling and diluted in radiolysis protecting solution. The data of the comparison is listed in Table 6-4.

TABLE 6-4
Stability comparison between Compound B and Compound
A over 3 days storage at RT (samples were diluted
with radiolysis protecting buffer post labeling in
a final radioactive concentration of ~11 mCi/mL)

| | |
|---|---|
| Compound A | Compound B |

| | 0 day | 3 day | 0 day | 3 day |
|---|---|---|---|---|
| RCP (%) | 97.9 | 96.3 | 89.5 | 88.6 |
| Identified | 0 | 0 | 10.3 | 8.8 |
| impurity (%) | | | | |
| Free ¹³¹I (%) | 0 | 2.5 | 0.1 | 2.4 |

The T1(TFA)₃/TFA method could be used in ¹³¹I-labeling for Compound C, Compound D and Compound A. Different from Compound B, ¹³¹I-Compound C, ¹³¹I-Compound D and ¹³¹I-Compound A were stable and there was almost no degradation occurring during the radioiodination.

There was no stability difference between Compound B and Compound A post labeling and then diluted in radiolysis protecting solution (degradation of Compound B only appeared during the radioiodination with presence of oxidant, such as T1(TFA)₃).

### Example 7

### Comparison of the Solubility of Compound C, Compound D & Compound A

Purpose: Solubility testing for Compound C, Compound D and Compound A at a concentration of 3 mg/mL.

### Example 7-A

### Significant Difference in Aqueous Solubility was Evidenced Among Compound C, Compound D, and Compound A

The compounds were dissolved in PEG400 and ethanol first and then diluted in matrix into a final concentration of 3 mg/mL. The components of the solution matrix included 3% sodium gentisate (w/v) and 6% ascorbic acid (w/v) (pH=3.6). The prepared sample solution was kept at RT and examined by visual inspection. The results are listed in the Table 7-1.

TABLE 7-1
Solubility comparison among Compound A, Compound D and Compound C (3
mg/mL) dissolved in PEG400/ethanol first and then diluted in matrix

| | Compound A | | Compound D | | Compound C | |
|---|---|---|---|---|---|---|
| Concentration | 3 mg/mL | | 3 mg/mL | | 3 mg/mL | |
| Component | 30% | PEG400 | 30% | PEG400 | 30% | PEG400 |
| | 2% | ethanol | 2% | ethanol | 2% | ethanol |
| | 6% | ascorbic acid | 6% | ascorbic acid | 6% | ascorbic acid |
| | 3% | sodium gentisate | 3% | sodium gentisate | 3% | sodium gentisate |

| | | | |
|---|---|---|---|
| Solution | Clear solution | Immediate | Immediate |
| inspection | | precipitation | precipitation |

Also, the solubility comparison at pH=4.3 also showed similar results as indicated above.

Example 7-B

### Addition of Acetic Acid Enhanced the Solubility for Compound C and Compound D

The compounds were dissolved in acetic acid first, and then diluted with PEG400/ethanol; the solution was finally diluted in matrix into a final concentration of 3 mg/mL. The components of the matrix included 3% sodium gentisate (w/v) and 6% ascorbic acid (w/v) (pH=3.6). The prepared sample solution was kept at RT and examined by visual inspection. The results are listed in the Table 7-2.

TABLE 7-2
Solubility comparison between Compound D and Compound
C (3 mg/mL) dissolved in acetic acid, then PEG400/ethanol,
and finally diluted in matrix

| | Compound D | | Compound C | |
|---|---|---|---|---|
| Concentration | 3 mg/mL | | 3 mg/mL | |
| Component | 3% | acetic acid | 3% | acetic acid |
| | 30% | PEG400 | 30% | PEG400 |
| | 2% | ethanol | 2% | ethanol |
| | 6% | ascorbic acid | 6% | ascorbic acid |
| | 3% | sodium gentisate | 3% | sodium gentisate |

| | | |
|---|---|---|
| Solution | Clear solution | Clear solution |
| inspection | | |

### Example 7-C

### Solubility Comparison Among Compound A, Compound C and Compound D Over One Week Storage at RT

Compound C and Compound D were dissolved in acetic acid first, and then with PEG400/ethanol; the solution was finally diluted in matrix into a final concentration of 3 mg/mL. The matrix included 3% sodium gentisate (w/v) and 6% ascorbic acid (w/v) (pH=3.6). The prepared sample solution was kept at RT and examined by visual inspection. The concentration of the tested compounds in the solution or in the supernatant was analyzed by HPLC. The results are listed in the Table 7-3.

TABLE 7-3
Solubility comparison among Compound A, Compound D and Compound C (3 mg/mL)
over 1 week storage at RT; their concentration was confirmed by HPLC

| | Compound A* | | Compound D | | Compound C | |
|---|---|---|---|---|---|---|
| Concentration | 3 mg/mL | | 3 mg/mL | | 3 mg/mL | |
| Component | 30% | PEG400 | 3% | acetic acid | 3% | acetic acid |
| | 2% | ethanol | 30% | PEG400 | 30% | PEG400 |
| | 6% | ascorbic acid | 2% | ethanol | 2% | ethanol |
| | 3% | sodium gentisate | 6% | ascorbic acid | 6% | ascorbic acid |
| | | | 3% | sodium gentisate | 3% | sodium gentisate |
| Solution | Clear solution | | A few tiny particles | | Precipitation** | |
| inspection | | | at the bottom | | | |
| at 7 days | | | | | | |
| HPLC measured | 2.94 | mg/mL | 2.89 | mg/mL | 0.23 | mg/mL*** |
| conc. at 7 days | | | | | | |

*Without adding acetic acid;
**clear solution post preparation and then precipitation occurred at approximately 2 hrs post storage;
***concentration in the supernatant.

All three tested compounds (Compound C, Compound D and Compound A) showed poor aqueous solubility, and co-solvent PEG400 was required in solution preparation.

The order of aqueous solubility was Compound A>Compound D>>Compound C.

### Example 8

### Effect of Adding Acetic Acid on ¹²⁷I-Compound A Iodination

Purpose: Increase the volume and decrease to level of corrosion in ¹²⁷I-Compound A iodination.

TABLE 8-1
Experimental conditions
Step-1: Intermediate formation

| | |
|---|---|
| Compound A-p | 80 μg in TFA |
| Tl(TFA)₃ | 80 μg in TFA |
| Volume of TFA | 90 μL |
| Volume of acetic acid | 210 μL (70%, v/v) |
| Total volume | 300 μL |
| Incubation time for | 10 min at RT |
| intermediate | |

Step-2: Radioiodination

| | |
|---|---|
| Na¹³¹I | 12.5 μg (Compound A-p: ¹²⁷I~2.3:1) |
| Volume of 0.1N NaOH | 12.5 μL |
| Reaction Vial | 2-mL Hollister |
| Reaction time | 5 min at RT |
| Volume of protecting | 1 mL (6% PEG400; 6% ascorbic acid; |
| solution added | 3% sodium gentisate) |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |
| Compound A peak area | 42.1% |
| Compound A-p peak area | 57.9% |
| Other impurity | 0% |

Results: The addition of acetic acid in T1(TFA)₃/TFA reaction solution did not significantly impact the iodination of Compound A, and the reaction could be completed based on the calculation of molar ratio of Compound A-p and Na¹²⁷I applied.

### Example 9

### Effect of the Concentration of Precursor on Yield of ¹²⁷I-Compound A Iodination

Purpose: The effect of the concentration of precursor on the yield of 127I-Compound A iodination was evaluated by using the fixed amount of precursor with increased volume of TFA/acetic acid (1:2 in volume).

TABLE 9-1
Experimental conditions:
Step-1: Intermediate formation

| | |
|---|---|
| Compound A-p | 100 μg in acetic acid |
| Tl(TFA)₃ | 100 μg in TFA |
| Volume of TFA | 0.1, 0.2, or 0.4 mL |
| Volume of acetic acid | 0.2, 0.4, or 0.8 mL |
| Total volume | 0.3, 0.6, or 1.2 mL |
| Incubation time for | 10 min at RT |
| intermediate | |

Step-2: Radioiodination

| | |
|---|---|
| Na¹²⁷I | 10 μg |
| Volume of 0.1N NaOH | 50 μL |
| Reaction Vial | 2-mL Hollister |
| Reaction time | 5 min at RT |
| Volume of protecting | 1 mL (6% PEG400; 6% ascorbic acid; |
| solution added | 3% sodium gentisate) |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |

Results: The concentration of Compound A-p was critical and there was almost no labeling when the concentration of Compound A-p was at 0.08 mg/mL. The data of the comparison is listed in Table 9-2.

TABLE 9-2
Effect of concentration of Compound A-p
on the yield of Compound A drug product

| | Final concentration of | | | | | |
|---|---|---|---|---|---|---|
| | Compound A-p | | | | | |
| | 0.32 mg/mL | | 0.16 mg/mL | | 0.08 mg/mL | |
| TFA/acetic acid | 0.3 | mL | 0.6 | mL | 1.2 | mL |
| Compound A-p, 10 mg/mL in | 10 | μL | 10 | μL | 10 | μL |
| acetic acid | (100 μg) | | (100 μg) | | (100 μg) | |
| Tl(TFA)₃, 20 mg/mL in TFA | 5 | μL | 5 | μL | 5 | μL |
| NaI, 0.2 mg/mL in | 50 | μL | 50 | μL | 50 | μL |
| 0.1N NaOH | | | | | | |

| | | | |
|---|---|---|---|
| Compound A-p, (%) | 85% | 83% | 100% |
| peak area | | | |
| Compound A, (%) | 15% | 17% | 0% |
| peak area | | | |

### Example 10

### Effect of the Volume of Na¹²⁷I, dissolved in 0.1 N NaOH, on yield of ¹²⁷I-Compound A Iodination

Purpose: Evaluate the effect of the volume aqueous solution (Na¹²⁷I in 0.1 N NaOH) on the yield of ¹²⁷I-Compound A iodination.

TABLE 10-1
Experimental conditions
Step-1: Intermediate formation

| | |
|---|---|
| Compound A-p | 80 μg in acetic acid |
| Tl(TFA)₃ | 80 μg in TFA |
| Volume of TFA | 90 μL |
| Volume of acetic acid | 210 μL |
| Total volume | 300 μL |
| Incubation time for | 10 min at RT |
| intermediate | |

Step-2: Radioiodination

| | |
|---|---|
| Na¹²⁷I | 25 μg |
| Volume of 0.1N NaOH | 0, 25, 50, or 100 μL |
| Reaction Vial | 2-mL Hollister |
| Reaction time | 5 min at RT |
| Volume of protecting | 1 mL (6% PEG400; 6% ascorbic acid; |
| solution added | 3% sodium gentisate) |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |

Results: The data is listed in Table 10-2, which demonstrated that the iodination of Compound A was preferred in acetic acid/TFA organic solution, and the yield was decreased over the volume of aqueous solution (0.1 N NaOH) applied.

TABLE 10-2
Effect of volume of NaI solution on
the yield of Compound A drug product

| | 12.5 μL | | 25 μL | | 50 μL | | 100 μuL | |
|---|---|---|---|---|---|---|---|---|
| TFA/acetic acid | 0.3 | mL | 0.3 | mL | 0.3 | mL | 0.3 | mL |
| Compound A-p, 10 mg/mL in | 8 | μL | 8 | μL | 8 | μL | 8 | μL |
| acetic acid | | | | | | | | |
| Tl(TFA)₃, 20 mg/mL in TFA | 4 | μL | 4 | μL | 4 | μL | 4 | μL |
| NaI, | 25 | μg | 25 | μg | 25 | μg | 25 | μg |
| In 0.1N NaOH | 12.5 | μL | 25 | μL | 50 | μL | 100 | μL |

| | | | | |
|---|---|---|---|---|
| Compound A-p, (%) | 57.9% | 74.0% | 79.2% | 100% |
| peak area | | | | |
| Compound A, (%) | 42.1% | 26.0% | 20.8% | 0% |
| peak area | | | | |

### Example 11

### Compound A Formulation with 70 and 250 mCi of Na¹³¹I

Purpose: Check the feasibility and stability of Compound A formulated with escalated dose, 70 or 250 mCi, of Na¹³¹I.

### Example 11-A

### Compound A Formulation with 70 mCi of Na¹³¹I

TABLE 11-1
Experimental conditions
Step-1: Intermediate formation

| | |
|---|---|
| Compound A-p | 55 μg in acetic acid |
| Tl(TFA)₃ | 92 μg in TFA |
| Volume of TFA | 100 μL |
| Volume of acetic acid | 100 μL |
| Total volume | 200 μL |
| Incubation time for | 10 min at RT |
| intermediate | |

Step-2: Radioiodination

| | |
|---|---|
| Na¹³¹I | 72 mCi |
| Volume of 0.1N NaOH/ | 20 μL |
| 0.02M Na₂SO₄ | |
| Reaction Vial | 2-mL (R-02, Na¹³¹I source vial Nordion) |
| Reaction time | 5 min at RT |
| Volume of protecting | 1 mL (6% PEG400; 6% ascorbic acid; |
| solution added | 3% sodium gentisate) |
| RP-HPLC analysis | Eclipse plus C18 (4.6 × 150 mm; |
| | 3.5 μm) |
| | Gradient: 10-50% B@15 min |
| | Solvent A: 0.1% TFA/H₂O; |
| | Solvent B: ethanol |
| | Column tem: 30° C. |
| | Flow rate: 1.5 mL/min |

### Example 11-B

### Compound A Formulation with 250 mCi of Na¹³¹I

TABLE 11-2
Experimental conditions
Step-1: Intermediate formation

| | |
|---|---|
| Compound A-p | 196 μg in acetic acid |
| Tl(TFA)₃ | 330 μg in TFA |
| Volume of TFA | 200 μL |
| Volume of acetic acid | 200 μL |
| Total volume | 400 μL |
| Incubation time for | 10 min at RT |
| intermediate | |

Step-2: Radioiodination

| | |
|---|---|
| Na¹³¹I | 250 mCi |
| Volume of 0.1N NaOH/ | 30 μL |
| 0.02M Na₂SO₄ | |
| Reaction Vial | 2-mL (R-02, Na¹³¹I source vial Nordion) |
| Reaction time | 5 min at RT |
| Volume of protecting | 1 mL (6% PEG400; 6% ascorbic acid; |
| solution added | 3% sodium gentisate) |
| RP-HPLC analysis | Eclipse plus C18 (4.6 × 150 mm; |
| | 3.5 μm) |
| | Gradient: 10-50% B@15 min |
| | Solvent A: TFA/H₂O; |
| | Solvent B: ethanol |
| | Column tem: 30° C. |
| | Flow rate: 1.5 mL/min |

Results: In this testing the potential of dose escalation in the optimized Compound A iodination method was preliminary evaluated. A molar ratio of 1.2 of T1(TFA)₃ to Compound A-p was applied in an intermediate reaction with a 10 min incubation at RT, which was then transferred into the radioactivity source vial, mixed and incubated with an additional 5 min. The results demonstrated that there was no significant change in radioactive impurity profile when a large dose of 250 mCi was applied in Compound A formulation, with an RCY of >95% (Table 11-3).

TABLE 11-3
Summary of preliminary testing for the dose
escalation of Compound A formulation

| | |
|---|---|
| 70 mCi | 250 mCi |
| n = 3 | n = 2 |

| | | | | |
|---|---|---|---|---|
| TFA/acetic acid | 0.2 | mL | 0.2 | mL |
| Compound A-p | 55 | μg | 196 | μg |
| Tl(TFA)₃ | 92 | μg | 330 | μg |

| | | |
|---|---|---|
| Na¹³¹I | 70 mCi (20 μL) | 250 mCi (30 μL) |
| RCY (%) | 97.6; 98.0; 94.4 | 96.2; 96.5 |
| Free ¹³¹I (%) | 0.6; 1.2; 0.3 | 0.5; 0.5 |

### Example 12

### Determination of Components in Excipient Used for Compound A Formulation

Purpose: Evaluate and finalize the components of excipient used for Compound A formulation to maintain the solubility and stability of the drug product.

### Example 12-A

### Effect of pH on the Solubility of Compound A

A solution of Compound A at a concentration of 3 mg/mL was requested for the tentative plan of acute toxicity testing. The sample solution was prepared by dissolving the Compound A drug substance in PEG400 first and then diluted in matrix with a pH range of 4.05-4.95, yielding a final PEG400 concentration of 30% (w/v). The component of the matrix included 3% sodium gentisate (w/v) and 6% ascorbic acid (w/v). The pKa of ascorbate is 4.17, and the pH of matrix solution was adjusted by mixing different portions of sodium ascorbate and ascorbic acid, generating a total ascorbic acid of 6% (sodium salt form plus free acid form). The solubility of the prepared sample solution was kept at RT and examined by visual inspection. The results are listed in the Table 12-1.

TABLE 12-1
Solubility of Compound A drug substance (3 mg/mL) dissolved
in PEG400 and then diluted in different pH of matrix.

| | pH 4.95 | pH 4.60 | pH 4.35 | pH 4.05 |
|---|---|---|---|---|
| | Matrix | Matrix | Matrix | Matrix |
| Con. | 3 mg/mL | 3 mg/mL | 3 mg/mL | 3 mg/mL |
| Compound A | | | | |
| % of PEG400 | 30% | 30% | 30% | 30% |
| Solution | Precipitated | Clear solution | Clear solution | Clear solution |
| inspection | within 3 hr | over 24 hr | over 24 hr | over 24 hr |

### Example 12-B

### Solubility of Compound A in PEG400

Long term solubility testing of Compound A was performed at a concentration of 0.8 mg/mL or 16 μg/mL. Compound A drug substance was dissolved in PEG400 first and then diluted in matrix, generating a different % of PEG400 in the final solution. The component of the matrix included 3% sodium gentisate (w/v), 2% ethanol (v/v), and 6% ascorbic acid (w/v) (3.7% sodium ascorbate and 2.7% ascorbic acid) in SWFI with a final pH of 4.3. The solubility of the prepared sample solution was examined by visual inspection and confirmed by the HPLC analysis. The results are listed in the Table 12-2.

TABLE 12-2
Addition of PEG400 to increase the solubility of Compound A drug substance.

| | 6% | 8% | 10% | 12% | 6% |
|---|---|---|---|---|---|
| | PEG400 | PEG400 | PEG400 | PEG400 | PEG400 |
| Con. | 0.8 mg/mL | 0.8 mg/mL | 0.8 mg/mL | 0.8 mg/mL | 16 μg/mL |
| Compound A | | | | | |
| Solubility | Clear | Clear | Clear | Clear | Clear |
| inspection | solution | solution | solution | solution | solution |
| | first; | first; | over one | over one | over one |
| | precipitated | precipitated | week | week | month |
| | within 4 hr | within 20 hr | storage | storage | storage |

Results: Based on the observations, the final components of excipient used for Compound A drug product formulation is listed below:
6% PEG400 (w:w)
2% ethanol (v/v)
3% sodium gentisate (w/v)
6% ascorbic acid (w/v) (3.7% sodium ascorbate and 2.7% ascorbic acid)
pH of 4.4±0.3

### Example 13

### Selection of Syringe Filter to Maximize the Recovery of Compound a Drug Product

Purpose: Evaluate, compare and select sterile syringe filter to maximize the radioactivity recovery.

A Compound A mixture solution used for this testing was prepared by mixing 0.5 mL (˜1.2 mCi) with 30 mL excipient (6% PEG400, 3% sodium gentisate and 6% ascorbic acid, pH=4.3). An aliquot of 4 mL sample solution was applied per filter testing and the solution was eluted with a 10 mL vacuum vial. The radioactivity remained on the filter and recovered in the collected solution was measured; the percentage of the radioactivity slicked on the membrane was calculated. The results are listed in the Table 13-1.

TABLE 13-1
The evaluation and selection of syringe filter for Compound A drug product aseptic filtration.

| | Filter: | | | | | | | |
|---|---|---|---|---|---|---|---|---|
| | | | | | | | Millex | Millex |
| | 17845DCK | 17764K | 16532K | 16596HYK | 17528 | 17575K | GS | GV |
| Hold vol. | 0.15 mL | 0.15 mL | 0.1 mL | 0.25 mL | N/A | 0.1 mL | N/A | N/A |
| Pore size | 0.2 um | 0.2 um | 0.2 um | 0.2 um | 0.2 um | 0.2 um | 0.22 um | 0.22 um |
| membrane | Nylon | Cellulose | PES | PTFE | SFCA | PTFE | MCE | PVDF |
| Membrane | 33% | 14%** | 38% | N/A* | 26% | N/A* | 97% | 23%*** |
| stick (%) | | | | | | | | |

*The resistance was too high and the sample solution did not easily filter through the membrane.
**No sterile form of this product commercially available.
***The stick could be significantly decreased by pre-wet the membrane and/or with an increased concentration of Compound A.

### Example 14

### Stability of Compound A Drug Product (1.25 mCi/mL) at Different Temperature, RT vs. −80° C.

Purpose: Evaluate and compare the stability of Compound A (1.25 mCi/mL) drug product stored at different temperature.

The stability of Compound A drug product in excipient [6% PEG400 (w/v), 3% sodium gentisate (w/v), 2% ethanol (v/v), 3.7% sodium ascorbate (w/v), and 2.7% ascorbic acid(w/v) in SWFI, with a final pH of 4.3] was examined and compared over the storage at different temperature.

Approximately 42 mCi of Compound A crude reaction solution was injected into the HPLC, and 33.6 mCi of the Compound A drug product was collected. The volatile organic in the drug product collection was removed by heating the vial at 70° C. for 60 min. Compound A drug product was diluted in excipient to a final radioactive concentration of 1.25 mCi/mL at TOC and then dispensed into 2-mL vials. The samples were stored at RT or in freezer (−80 ° C.). The RCP was tested at pre-determined time points, and the results are listed in Table 14-1.

TABLE 14-1
Stability of Compound A drug product (1.25 mCi/mL, 2 mL per vial)
over 7 days storage at either RT or −80° C.

| | 0-day* | | | | | | | |
|---|---|---|---|---|---|---|---|---|
| | Before | After | 1-day | | 3-day | | 7-days | |
| | drying | drying | RT | −80° C. | RT | −80° C. | RT | −80° C. |
| RCP (%) | N/A | 92.7 | 87.4 | 92.7 | 83.8 | 92.7 | 81.5 | 92.7 |
| Free ¹³¹I | N/A | 9.3 | 12.6 | 9.3 | 16.2 | 9.3 | 18.5 | 9.3 |
| (%) | | | | | | | | |

*The drug product in HPLC collected solution was not protected during the heating process.

Results: Compound A drug product was stable over one week storage in freezer (−80° C.); while free ¹³¹I increased from 9.3% to 18.5% at 0 and 7 days post storage at RT, respectively.

### Example 15

### Stability Comparison of Compound A Drug Product (10 mCi/mL) stored at RT vs. −80° C.

Purpose: Evaluate and compare the stability of Compound A drug product with a radioactivity concentration of 10 mCi/mL at different temperature: RT vs. −80° C.

Approximately 60 mCi of Compound A crude reaction solution was injected into the HPLC, and 43.8 mCi of the Compound A drug product was collected. The volatile organic on the drug product collection was removed by heating the vial at 70° C. for 60 min. The Compound A was diluted in excipient to a final radioactive concentration 10 mCi/mL and then dispensed into 2-mL vials. The samples were stored either at RT or in freezer (−80° C.). The RCP was tested at pre-determined time points, and the results are listed in Table 15-1.

TABLE 15-1
Stability of Compound A drug product (10 mCi/mL) over
8 d storage at either RT or −80° C.

| | 0-day* | | | | | | | |
|---|---|---|---|---|---|---|---|---|
| | Before | After | 1-day | | 3-day | | 8-days | |
| | drying | drying | RT | −80° C. | RT | −80° C. | RT | −80° C. |
| RCP(%) | 99.4 | 98.3 | 94.3 | 97.6 | 89.2 | 97.2 | 85.0 | 95.8 |
| Free ¹³¹I | 0.4 | 1.5 | 5.7 | 2.0 | 10.8 | 2.8 | 15.0 | 4.2 |
| (%) | | | | | | | | |

*Compound A HPLC collection was protected by excipient during the heating process.

Results: The free ¹³¹I was <5% for Compound A drug product stored in freezer (−80° C.) for 8 days; while free ¹³¹I increased from 1.5% to 15.0% at 0 and 8 days post storage at RT, respectively.

### Example 16

### Organ Distribution and Tumor Accumulation of ¹³¹I-Cmpd A, ¹³¹I-Cmpd B, ¹³¹I-Cmpd C, and ¹³¹I-Cmpd D in mice

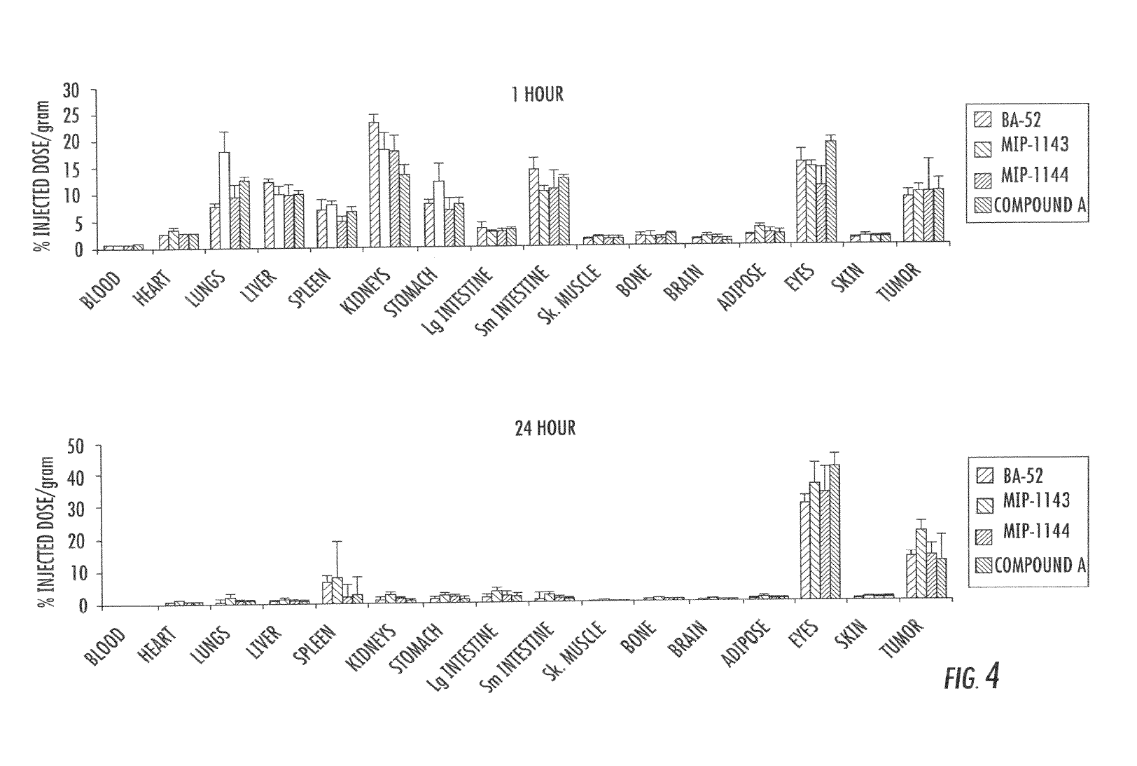

The organ distribution and tumor accumulation experiments were performed in B16F10 tumor bearing mice. At times indicated (1 hour and 24 hour) after intravenous administration of the substances, the animals were sacrificed, organs and tumors were removed, optionally dabbed dry, weighed, and measured for radioactive content in a calibrator with the corresponding isotope standard. The results are depicted as % of the injected dose/gm of tissue. FIG. 4 illustrates the biodistribution of ¹³¹I-Cmpd A, ¹³¹I-Cmpd B, ¹³¹I-Cmpd C, and ¹³¹I-Cmpd D in B16F10 tumor bearing mice. The graphs indicate that desirable distribution properties were observed in all four tested compounds.

### Example 17

### Effect of ¹³¹I-Cmpd A on SK-MEL-3 Tumor Growth in Mice

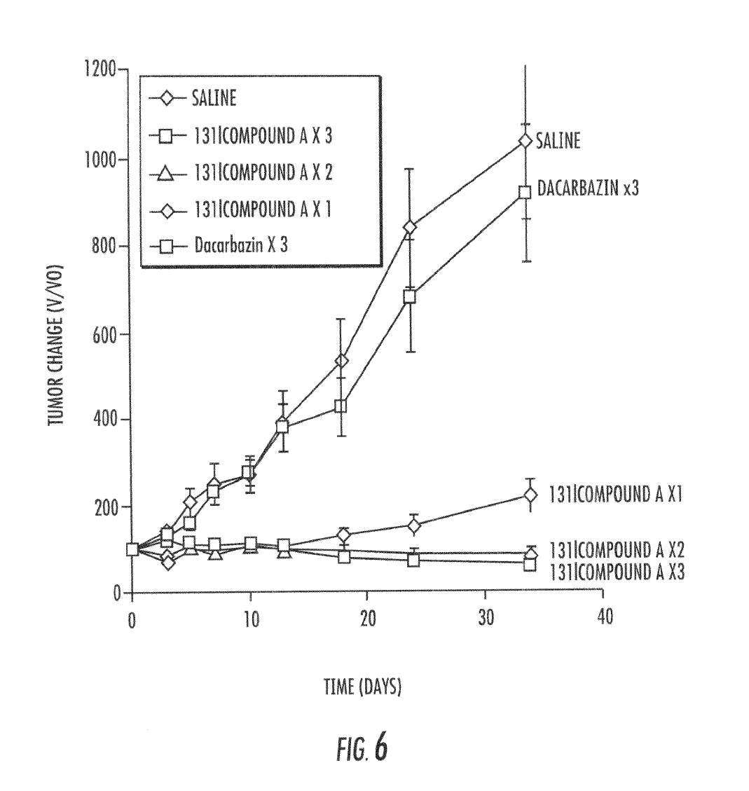

Effects of ¹³¹I-Cmpd A (68 mCi/m²) on SK-MEL-3 tumor growth were investigated in mice. Saline and dacarbazine were used as references for the study. In this experiment, different batches of mice were administered a dose of 68 mCi/m² of ¹³¹I-Cmpd A once a day, twice a day, and thrice a day. The treatment lasted for 125 days. The mice were euthanized if tumor volume was greater than 1500 mm³. The treated mice were closely monitored and sacrificed if any signs of approaching death were shown. Tumor change (length and width of tumor) was monitored every few days. The tumor change was quantitatively measured and the results shown in FIG. 6 indicate the effectiveness of ¹³¹I-Cmpd A in reducing the tumor growth.

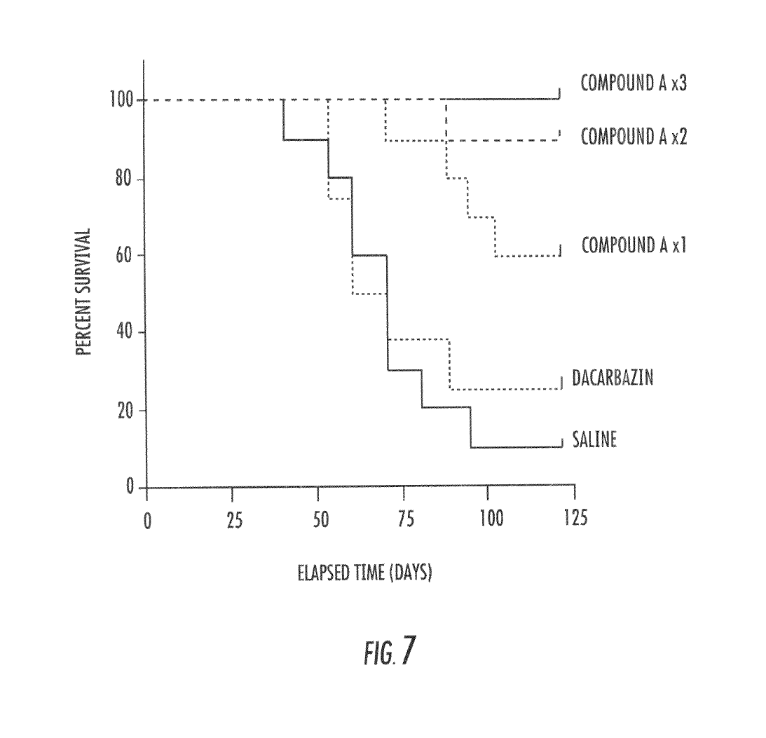

Also, the results shown in FIG. 7 indicate the effectiveness of ¹³¹I-Cmpd A in increasing survival of the mice.

### Example 18

### Synthesis of N-(2-diethylamino-ethyl)-4-(4-flouoro-benzoylamino)-5-iodo-2-methoxy-benzamide (Compound A)

The title compound may be synthesized by methods known in the art analogous to the synthesis demonstrated in WO 2005/089815, which is hereby incorporated by reference in its entirety.

### Example 19

### Synthesis of Benzo[1,3]dioxole-5-carboxylic acid [4-2-diethylamino-ethylcarbamoyl)-2-iodo-5-methoxyphenyl]amide (Compound B)

The title compound may be synthesized by methods known in the art analogous to the synthesis demonstrated in WO 2005/089815, which is hereby incorporated by reference in its entirety.

### Example 20

### Synthesis of 4-(4-chloro-benzoylamino)-N-(2-diethylamino-ethyl)-5-iodo-2-methoxy-benzamide (Compound C)

The title compound may be synthesized by methods known in the art analogous to the synthesis demonstrated in WO 2005/089815, which is hereby incorporated by reference in its entirety.

### Example 21

### Synthesis of N-(2-diethylamino-ethyl)-5-iodo-2-methoxy-4-(4-methyoxy-benzoylamino)-benzamide (Compound D)

The title compound may be synthesized by methods known in the art analogous to the synthesis demonstrated in WO 2005/089815, which is hereby incorporated by reference in its entirety.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

1. A pharmaceutical composition comprising:
a compound of formula I: 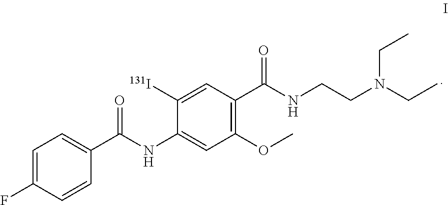
at least one solubilizer; and
at least one preservative;
wherein the composition has a pH of from about 4.0 to about 4.8.

2. The composition of claim 1 comprising about 1.25 mCi/milliter of the compound of formula I.

3. The composition of claim 1 comprising:
about 6% polyethylene glycol (w/v);
about 2% ethanol (v/v);
about 3% sodium gentisate (w/v); and
about 6% ascorbic acid (w/v).

4. The composition of claim 1 wherein the ascorbic acid is about 3.7% sodium ascorbate and 2.7% ascorbic acid.

5. The composition of claim 1 further comprising N-(2-(diethylamino)ethyl)-4-(4-fluorobenzamido)-5-iodo-2-methoxybenzamide.

6. A method of preparing a compound of formula III: 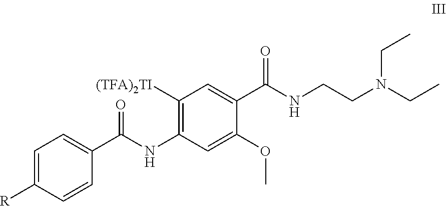
wherein R is fluoro, chloro or methoxy, said method comprising
contacting under reaction conditions a compound of formula II: 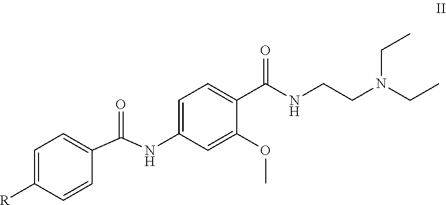
with a solution comprising at least about 1 equivalent of tris(2,2,2-trifluoroacetyl)thallium and a 1:1 volume of trifluoroacetic acid and acetic acid, to provide a compound of formula III.

7. A method of preparing a compound of formula I: 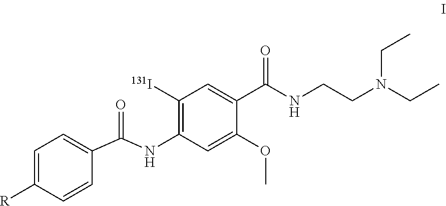
wherein R is fluoro, chloro or methoxy, comprising
contacting under first reaction conditions a compound of formula II: 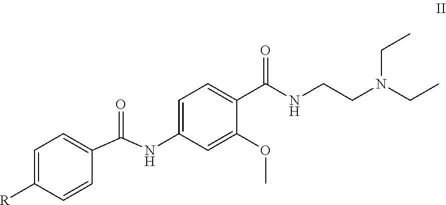
with a solution comprising at least about 1 equivalent of tris(2,2,2-trifluoroacetyl)thallium and a 1:1 volume ratio of trifluoroacetic acid and acetic acid, to provide a compound of formula III: 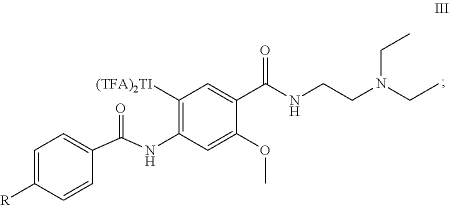
and
contacting under second reaction conditions a compound of formula III with a solution comprising at least about 0.5 to about 0.7 equivalents of sodium ¹³¹iodide, to provide a compound of formula I.

8. The method of claim 6, wherein the compound of formula II is present in a solution comprising acetic acid.

9. The method of claim 6, wherein the tris(2,2,2-trifluoroacetyl)thallium is present in a solution comprising trifluoroacetic acid.

10. The method of claim 6, wherein the sodium ¹³¹iodide is in a solution comprising sodium hydroxide and sodium sulfate.

11. The method of claim 6, wherein the reaction conditions comprise a reaction time of about fifteen minutes at about 25 degrees Celsius.

12. The method of claim 7, wherein the first reaction conditions comprise a reaction time of about ten minutes at about 25 degrees Celsius.

13. The method of claim 7, wherein the second reaction conditions comprise a reaction time of about five minutes at about 25 degrees Celsius.

14. The method of claim 7, further comprising isolating the compound of formula I.

15. The method of claim 14, wherein the isolating comprises high-performance liquid chromatography.

16. A method of treating a patient suffering from melanoma by administering a pharmaceutically effective amount of a pharmaceutical composition of claim 1.

17. The method of claim 7, wherein the compound of formula II is present in a solution comprising acetic acid.

18. The method of claim 7, wherein the tris(2,2,2-trifluoroacetyl)thallium is present in a solution comprising trifluoroacetic acid.

19. The method of claim 7, wherein the sodium ¹³¹iodide is in a solution comprising sodium hydroxide and sodium sulfate.
